(19) United States
(12) Patent Application Publication
(10) Pub. No.: US 2018/0239945 A1
(43) Pub. Date: Aug. 23, 2018

(54) FINGERPRINT IDENTIFICATION MODULE AND MANUFACTURING METHOD THEREOF

(71) Applicant: Primax Electronics Ltd., Taipei (TW)

(72) Inventors: MAO-HSIU HSU, Taipei (TW); KUAN-PAO TING, Taipei (TW)

(21) Appl. No.: 15/627,041

(22) Filed: Jun. 19, 2017

(30) Foreign Application Priority Data
Feb. 17, 2017 (TW) 106105293

(51) Int. Cl. G06K 9/00; A61B 5/1172

(52) U.S. Cl. CPC G06K 9/00013; G06K 9/0012; G06K 9/0004; G06K 9/00053; G06K 9/00087; A61B 5/1172

(57) ABSTRACT

A fingerprint identification module includes a substrate, a fingerprint sensor and a covering plate. The fingerprint sensor is disposed on the substrate and electrically connected with the substrate. The fingerprint sensor detects a fingerprint image. The covering plate is located over the fingerprint sensor and combined with the fingerprint sensor through an adhesive layer. The adhesive layer is arranged between the covering plate and the fingerprint sensor, and includes a solid adhesive part and a liquid adhesive part. The liquid adhesive part is arranged around the solid adhesive part.

### FIELD OF THE INVENTION

The present invention relates to an identity identification element, and more particularly to a fingerprint identification module for identifying the identity of a user through a fingerprint.

### BACKGROUND OF THE INVENTION

Recently, a fingerprint identification technology has been applied to a variety of electronic products. By using the fingerprint identification technology, the user's fingerprint can be inputted into an electronic product and saved in the electronic product. For unlocking the electronic product, the user has to input the fingerprint through a fingerprint identification module. The way of unlocking the electronic product by the fingerprint identification technology is faster and more user-friendly than the way of manually inputting the password. Consequently, the fingerprint identification technology is favored by many users, and the demands on the fingerprint identification module are gradually increased.

A structure of a conventional fingerprint identification module will be described as follows. FIG. 1 schematically illustrates a structure of a conventional fingerprint identification module. As shown in FIG. 1, the conventional fingerprint identification module 1 comprises a circuit board 11, a fingerprint sensor 12 and a covering plate 14. The fingerprint sensor 12 is disposed on the circuit board 11. Moreover, the fingerprint sensor 12 is electrically connected with the circuit board 11 to acquire electric power from the circuit board 11. The covering plate 14 is combined with the fingerprint sensor 12 through an adhesive layer 13. The covering plate 14 is used as a contact interface between the user's finger and the fingerprint sensor 12. Moreover, the covering plate 14 can protect the surface of the fingerprint sensor 12. Consequently, even if the fingerprint sensor 12 is frequently contacted with the user's finger, the surface of the fingerprint sensor 12 is not damaged. Moreover, the covering plate 14 has a specified color matching an electronic device (not shown) where the fingerprint identification module 1 is installed. When the user's finger is placed on the covering plate 14, the fingerprint image of the user's finger is sensed by the fingerprint sensor 12. The fingerprint image sensed by the fingerprint sensor 12 is transmitted to the electronic device through the circuit board 11 so as to be recognized.

However, the method of manufacturing the conventional fingerprint identification module still has some drawbacks. For example, during the feeding process, the fingerprint sensor is readily suffered from warpage. FIG. 2 schematically illustrates the combination of the fingerprint sensor and the covering plate of the conventional fingerprint identification module through the adhesive layer when the fingerprint sensor is suffered from warpage. As shown in FIG. 2, the fingerprint sensor 12′ is suffered from warpage. Moreover, the adhesive layer 13′ for combining the covering plate 14 and the fingerprint sensor 12′ is a solid adhesive (e.g., a film adhesive). After the fingerprint identification module 1 is fabricated, the fingerprint identification module 1 undergoes a reliability test such as a high temperature/high humidity test or a salt spray test. After the test, the periphery region of the junction between the covering plate 14 and the fingerprint sensor 12′ (e.g., the circumscribed region as shown in FIG. 2) is easily peeled off. In accordance with another conventional technology, a water-based adhesive is used as the adhesive layer 13 for combining the fingerprint sensor 12 and the covering plate 14. However, if only the water-based adhesive is used to combine the fingerprint sensor 12 and the covering plate 14, many bubbles are possibly generated in the middle region of the junction between the fingerprint sensor 12 and the covering plate 14. In other words, the conventional fingerprint identification module 1 still needs to be further improved.

### SUMMARY OF THE INVENTION

The present invention provides a fingerprint identification module and a manufacturing method of the fingerprint identification module. An adhesive layer of the fingerprint identification module includes a solid adhesive part and a liquid adhesive part. The liquid adhesive part is arranged around the solid adhesive part. Consequently, the performance of combining a covering plate and a fingerprint sensor of the fingerprint identification module is enhanced.

In accordance with an aspect of the present invention, there is provided a fingerprint identification module. The fingerprint identification module includes a substrate, a fingerprint sensor and a covering plate. The fingerprint sensor is disposed on the substrate and electrically connected with the substrate. The fingerprint sensor detects a fingerprint image. The covering plate is located over the fingerprint sensor and combined with the fingerprint sensor through an adhesive layer. The adhesive layer is arranged between the covering plate and the fingerprint sensor, and includes a solid adhesive part and a liquid adhesive part. The liquid adhesive part is arranged around the solid adhesive part.

In accordance with another aspect of the present invention, there is provided a manufacturing method of a fingerprint identification module. The manufacturing method includes the following steps. Firstly, a solid adhesive part is formed on a first one of a covering plate and a fingerprint sensor, and a liquid adhesive part is formed on a second one of the covering plate and the fingerprint sensor. There is a gap between the solid adhesive part and the first one of the covering plate and the fingerprint sensor. The liquid adhesive part is formed on a periphery region of the second one of the covering plate and the fingerprint sensor. Then, the covering plate is moved toward the fingerprint sensor, or the fingerprint sensor is moved toward the covering plate. Consequently, the covering plate and the fingerprint sensor are combined together, and the liquid adhesive part is arranged around the solid adhesive part.

In accordance with a further aspect of the present invention, there is provided a manufacturing method of a fingerprint identification module. The manufacturing method includes the following steps. Firstly, a solid adhesive part and a liquid adhesive part are formed on a specified one of a covering plate and a fingerprint sensor. There is a gap between the solid adhesive part a border of the specified one of the covering plate and the fingerprint sensor. The liquid adhesive part is disposed within the gap and arranged around the solid adhesive part. Then, the covering plate is moved toward the fingerprint sensor, or the fingerprint sensor is moved toward the covering plate. Consequently, the covering plate and the fingerprint sensor are combined together.

The above objects and advantages of the present invention will become more readily apparent to those ordinarily skilled in the art after reviewing the following detailed description and accompanying drawings, in which:

### BRIEF DESCRIPTION OF THE DRAWINGS

FIG. 1 schematically illustrates a structure of a conventional fingerprint identification module;

FIG. 2 schematically illustrates the combination of the fingerprint sensor and the covering plate of the conventional fingerprint identification module through the adhesive layer when the fingerprint sensor is suffered from warpage;

FIG. 3 schematically illustrates a structure of a fingerprint identification module according to an embodiment of the present invention;

FIG. 4 is a flowchart illustrating a method of manufacturing the fingerprint identification module according to a first embodiment of the present invention;

FIG. 5A schematically illustrates the concept of the step S11 of the manufacturing method as shown in FIG. 4;

FIG. 5B schematically illustrates the concept of the step S12 of the manufacturing method as shown in FIG. 4;

FIG. 5C schematically illustrates the concept of the step S13 of the manufacturing method as shown in FIG. 4;

FIG. 6 is a flowchart illustrating a method of manufacturing the fingerprint identification module according to a second embodiment of the present invention;

FIG. 7A schematically illustrates the concept of the step S21 of the manufacturing method as shown in FIG. 6;

FIG. 7B schematically illustrates the concept of the step S22 of the manufacturing method as shown in FIG. 6;

FIG. 7C schematically illustrates the concept of the step S23 of the manufacturing method as shown in FIG. 6;

FIG. 8 is a flowchart illustrating a method of manufacturing the fingerprint identification module according to a third embodiment of the present invention;

FIG. 9A schematically illustrates the concept of the step S31 of the manufacturing method as shown in FIG. 8;

FIG. 9B schematically illustrates the concept of the step S32 of the manufacturing method as shown in FIG. 8;

FIG. 9C schematically illustrates the concept of the step S33 of the manufacturing method as shown in FIG. 8;

FIG. 10 is a flowchart illustrating a method of manufacturing the fingerprint identification module according to a fourth embodiment of the present invention;

FIG. 11A schematically illustrates the concept of the step S41 of the manufacturing method as shown in FIG. 10;

FIG. 11B schematically illustrates the concept of the step S42 of the manufacturing method as shown in FIG. 10; and

FIG. 11C schematically illustrates the concept of the step S43 of the manufacturing method as shown in FIG. 10.

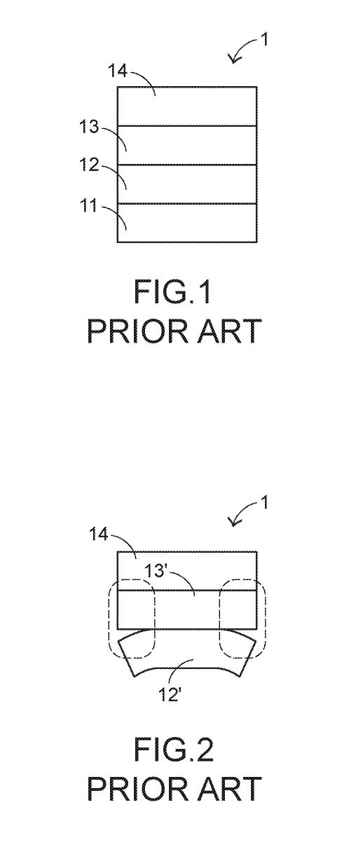

### DETAILED DESCRIPTION OF THE PREFERRED EMBODIMENT

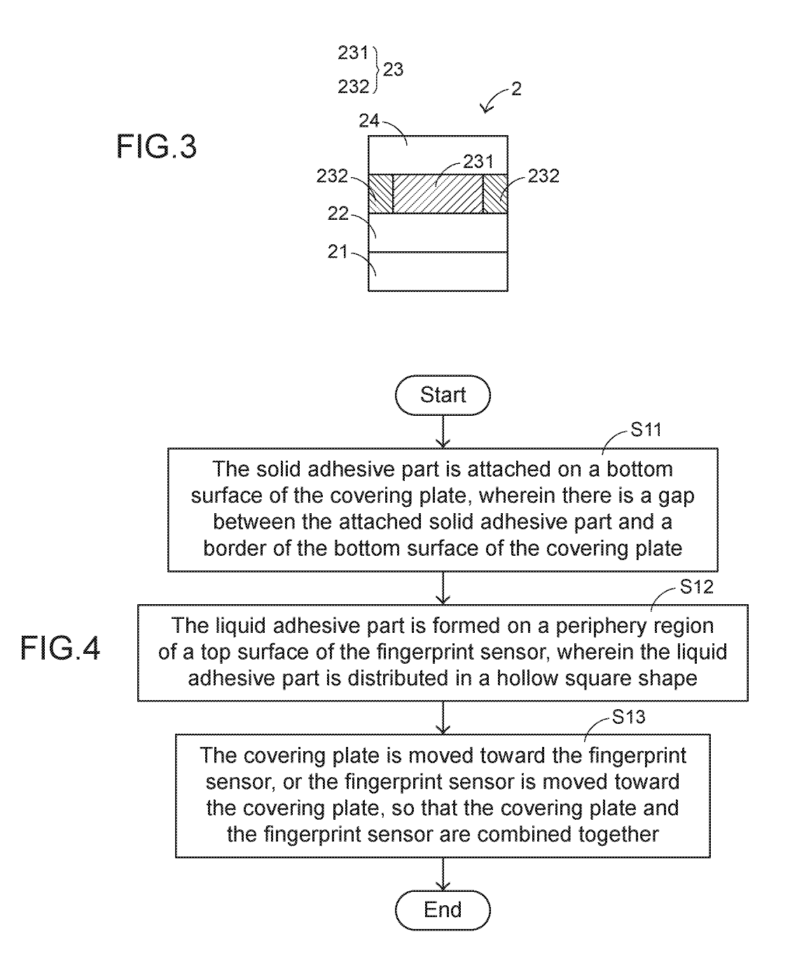

FIG. 3 schematically illustrates a structure of a fingerprint identification module according to an embodiment of the present invention. As shown in FIG. 3, the fingerprint identification module 2 comprises a substrate 21, a fingerprint sensor 22 and a covering plate 24. The fingerprint sensor 22 is disposed on the substrate 21. Moreover, the fingerprint sensor 22 is electrically connected with the substrate 21 to acquire electric power from the substrate 21. The covering plate 24 is located over the fingerprint sensor 22. The covering plate 24 is combined with the fingerprint sensor 22 through an adhesive layer 23. The covering plate 24 is used as a contact interface between the user's finger and the fingerprint sensor 22. Moreover, the covering plate 24 can protect the surface of the fingerprint sensor 22. Consequently, even if the fingerprint sensor 22 is frequently contacted with the user's finger, the surface of the fingerprint sensor 22 is not damaged. Moreover, the covering plate 24 has a specified color matching an electronic device (not shown) where the fingerprint identification module 2 is installed. When the user's finger is placed on the covering plate 24, the fingerprint image of the user's finger is sensed by the fingerprint sensor 22. The fingerprint image sensed by the fingerprint sensor 22 is transmitted to the electronic device through the substrate 21 so as to be recognized. Preferably but not exclusively, the substrate 21 is a flexible circuit board, the fingerprint sensor 22 is a fingerprint sensing chip, and the covering plate 24 is a glass plate or a ceramic plate.

Moreover, the adhesive layer 23 is arranged between the covering plate 24 and the fingerprint sensor 22. In this embodiment, the adhesive layer 23 comprises a solid adhesive part 231 and a liquid adhesive part 232. The liquid adhesive part 232 is arranged around the solid adhesive part 231. That is, the periphery region of the junction between the covering plate 24 and the fingerprint sensor 22 is the liquid adhesive part 232, and the middle region of the junction between the covering plate 24 and the fingerprint sensor 22 is the solid adhesive part 231. Preferably but not exclusively, the liquid adhesive part 232 is a water-based adhesive, and the solid adhesive part 231 is a film adhesive, and the liquid adhesive part 232 is a film adhesive.

As previously described, after the conventional fingerprint identification module 1 undergoes the high temperature/high humidity test or the salt spray test, the periphery region of the junction between the covering plate 14 and the fingerprint sensor 12 is suffered from warpage and easily peeled off because the solid adhesive is only used. Since the periphery region of the junction between the covering plate 24 and the fingerprint sensor 22 is the liquid adhesive part 232 according to the present invention, the attaching strength of the periphery region is enhanced. Especially when the fingerprint sensor 22 is possibly suffered from warpage, the liquid adhesive part 232 is effective to attach the periphery region of the fingerprint sensor 22 on the covering plate 24. As previously described, many bubbles are possibly generated in the middle region of the junction between the fingerprint sensor 12 and the covering plate 14 of the fingerprint identification module 1 because the liquid adhesive is only used. Since the middle region of the junction between the covering plate 24 and the fingerprint sensor 22 is the solid adhesive part 231 according to the present invention, the attaching uniformity in the middle region of the junction is enhanced. The enhanced attaching uniformity can avoid the generation of the bubbles. Consequently, the performance of the fingerprint identification module 2 to sense the fingerprint image is increased.

Some methods of manufacturing the fingerprint identification module 2 will be described as follows.

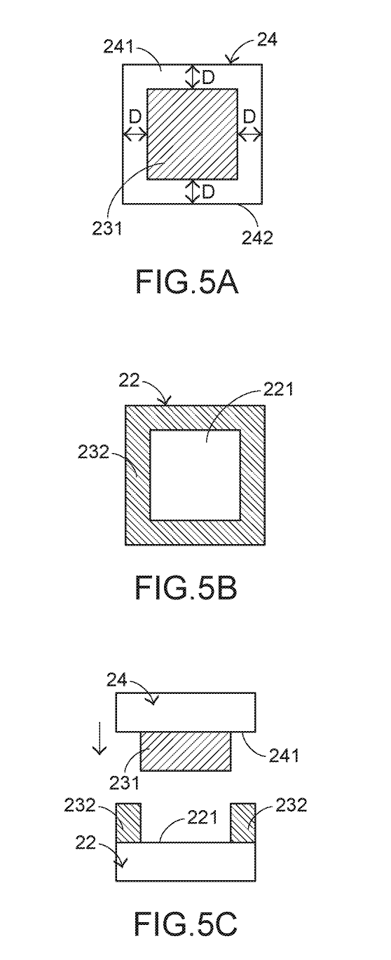

FIG. 4 is a flowchart illustrating a method of manufacturing the fingerprint identification module according to a first embodiment of the present invention. The manufacturing method comprises the following steps S11, S12 an S13. The concepts of these steps are shown in FIGS. 5A, 5B and 5C. In the step S11 and as shown in FIG. 5A, the solid adhesive part 231 is attached on a bottom surface 241 of the covering plate 24. Moreover, there is a gap D between the attached solid adhesive part 231 and a border 242 of the bottom surface 241 of the covering plate 24. In the step S12 and as shown in FIG. 5B, the liquid adhesive part 232 is formed on a periphery region of a top surface 221 of the fingerprint sensor 22. Moreover, the liquid adhesive part 232 is distributed in a hollow square shape. In the step S13 and as shown in FIG. 5C, the covering plate 24 is moved toward the fingerprint sensor 22, or the fingerprint sensor 22 is moved toward the covering plate 24. Consequently, the covering plate 24 and the fingerprint sensor 22 are combined together. Preferably but not exclusively, the gap D described in the step S11 and FIG. 5A is in the range between 1 mm and 2 mm. Moreover, according to the practical requirements, the step S11 and the step S12 may be exchanged.

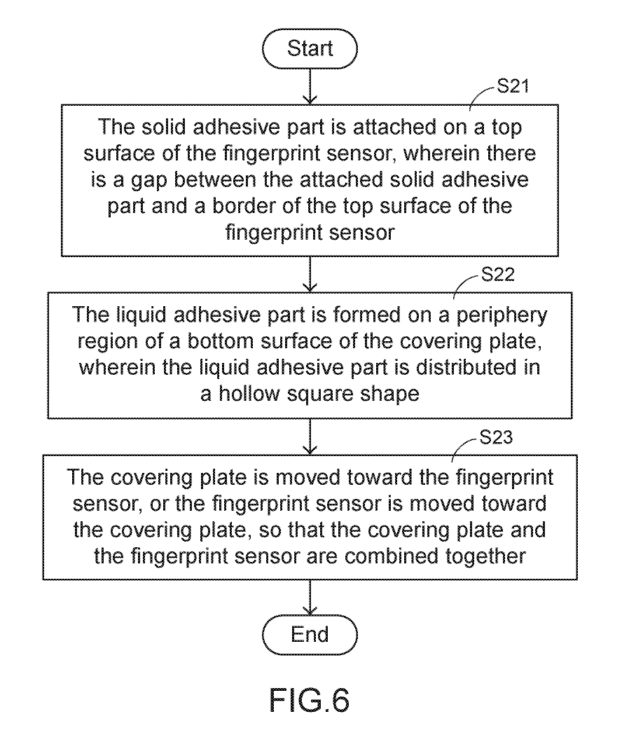

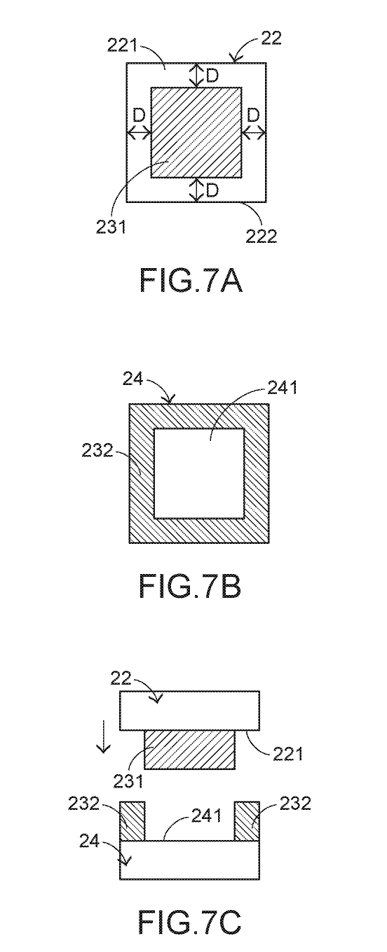

FIG. 6 is a flowchart illustrating a method of manufacturing the fingerprint identification module according to a second embodiment of the present invention. The manufacturing method comprises the following steps S21, S22 an S23. The concepts of these steps are shown in FIGS. 7A, 7B and 7C. In the step S21 and as shown in FIG. 7A, the solid adhesive part 231 is attached on a top surface 221 of the fingerprint sensor 22. Moreover, there is a gap D between the attached solid adhesive part 231 and a border 222 of the top surface 221 of the fingerprint sensor 22. In the step S22 and as shown in FIG. 7B, the liquid adhesive part 232 is formed on a periphery region of a bottom surface 241 of the covering plate 24. Moreover, the liquid adhesive part 232 is distributed in a hollow square shape. In the step S23 and as shown in FIG. 7C, the covering plate 24 is moved toward the fingerprint sensor 22, or the fingerprint sensor 22 is moved toward the covering plate 24. Consequently, the covering plate 24 and the fingerprint sensor 22 are combined together. Preferably but not exclusively, the gap D described in the step S21 and FIG. 7A is in the range between 1 mm and 2 mm. Moreover, according to the practical requirements, the step S21 and the step S22 may be exchanged.

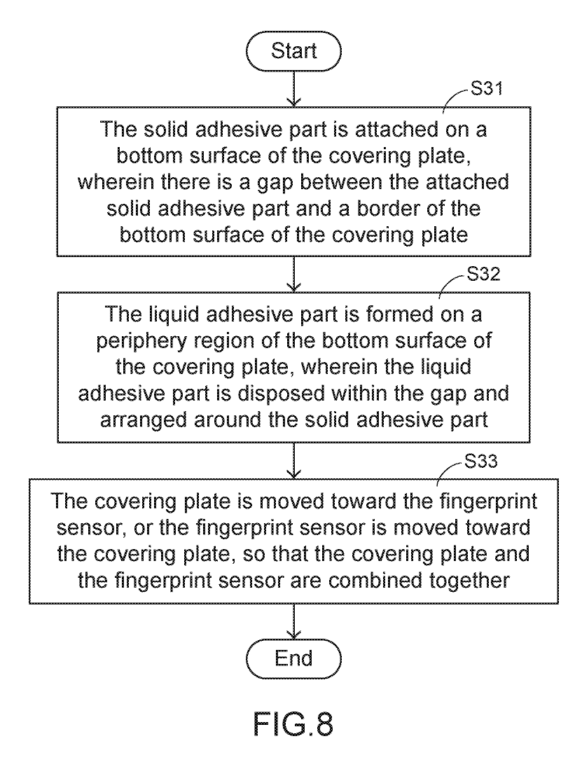

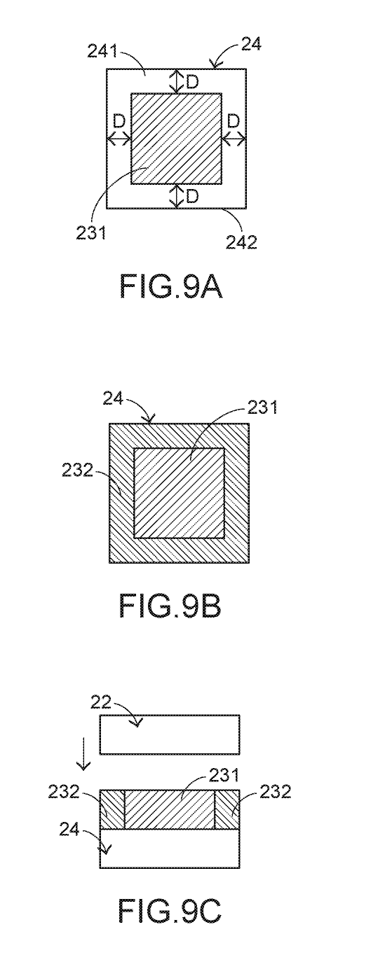

FIG. 8 is a flowchart illustrating a method of manufacturing the fingerprint identification module according to a third embodiment of the present invention. The manufacturing method comprises the following steps S31, S32 an S33. The concepts of these steps are shown in FIGS. 9A, 9B and 9C. In the step S31 and as shown in FIG. 9A, the solid adhesive part 231 is attached on a bottom surface 241 of the covering plate 24. Moreover, there is a gap D between the attached solid adhesive part 231 and a border 242 of the bottom surface 241 of the covering plate 24. In the step S32 and as shown in FIG. 9B, the liquid adhesive part 232 is formed on a periphery region of the bottom surface 241 of the covering plate 24. Moreover, the liquid adhesive part 232 is distributed in a hollow square shape. That is, the liquid adhesive part 232 is disposed within the gap D and arranged around the solid adhesive part 231. In the step S33 and as shown in FIG. 9C, the covering plate 24 is moved toward the fingerprint sensor 22, or the fingerprint sensor 22 is moved toward the covering plate 24. Consequently, the covering plate 24 and the fingerprint sensor 22 are combined together. Preferably but not exclusively, the gap D described in the step S31 and FIG. 9A is in the range between 1 mm and 2 mm. Moreover, according to the practical requirements, the step S31 and the step S32 may be exchanged.

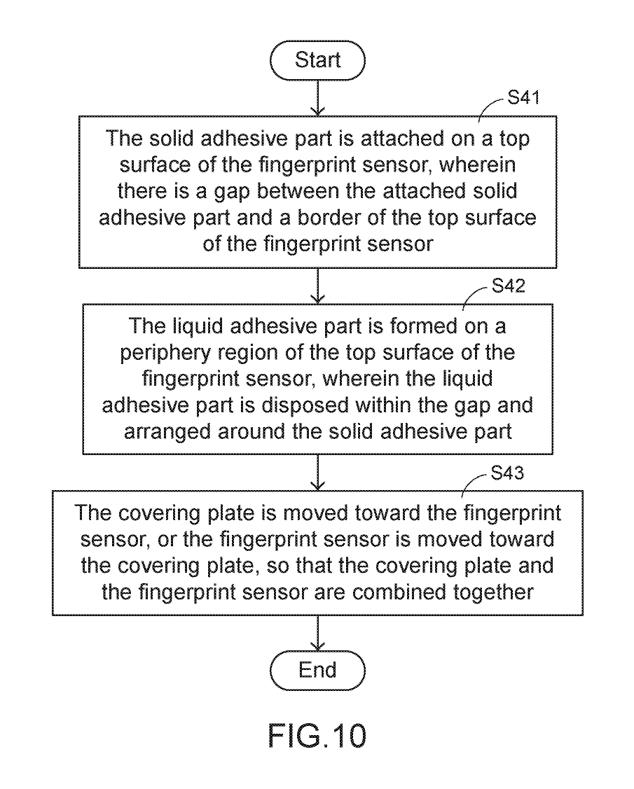

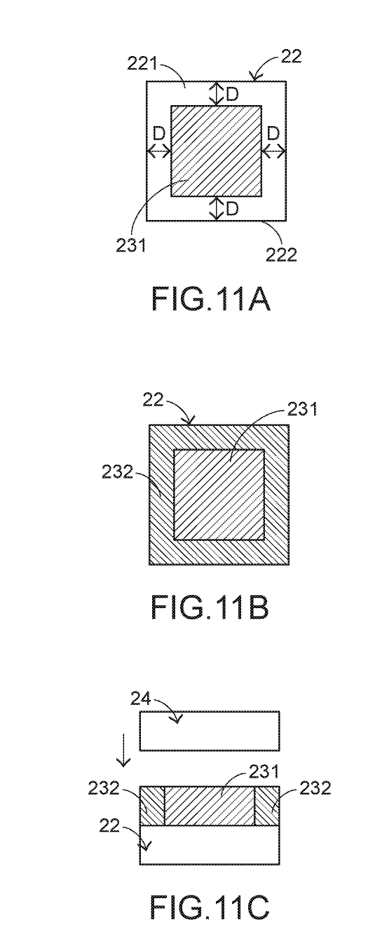

FIG. 10 is a flowchart illustrating a method of manufacturing the fingerprint identification module according to a fourth embodiment of the present invention. The manufacturing method comprises the following steps S41, S42 an S43. The concepts of these steps are shown in FIGS. 11A, 11B and 11C. In the step S41 and as shown in FIG. 11A, the solid adhesive part 231 is attached on a top surface 221 of the fingerprint sensor 22. Moreover, there is a gap D between the attached solid adhesive part 231 and a border 222 of the top surface 221 of the fingerprint sensor 22. In the step S42 and as shown in FIG. 11B, the liquid adhesive part 232 is formed on a periphery region of the top surface 221 of the fingerprint sensor 22. Moreover, the liquid adhesive part 232 is distributed in a hollow square shape. That is, the liquid adhesive part 232 is disposed within the gap D and arranged around the solid adhesive part 231. In the step S43 and as shown in FIG. 11C, the covering plate 24 is moved toward the fingerprint sensor 22, or the fingerprint sensor 22 is moved toward the covering plate 24. Consequently, the covering plate 24 and the fingerprint sensor 22 are combined together. Preferably but not exclusively, the gap D described in the step S41 and FIG. 11A is in the range between 1 mm and 2 mm. Moreover, according to the practical requirements, the step S41 and the step S42 may be exchanged.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all modifications and similar structures.

What is claimed is:

1. A fingerprint identification module, comprising:
a substrate;
a fingerprint sensor disposed on the substrate and electrically connected with the substrate, wherein the fingerprint sensor detects a fingerprint image; and
a covering plate located over the fingerprint sensor and combined with the fingerprint sensor through an adhesive layer, wherein the adhesive layer is arranged between the covering plate and the fingerprint sensor, and includes a solid adhesive part and a liquid adhesive part, wherein the liquid adhesive part is arranged around the solid adhesive part.

2. The fingerprint identification module according to claim 1, wherein before the covering plate is combined with the fingerprint sensor, the solid adhesive part is attached on a bottom surface of the covering plate, there is a gap between the solid adhesive part and a border of the bottom surface of the covering plate, the liquid adhesive part is formed on a top surface of the fingerprint sensor, and the liquid adhesive part is formed on a periphery region of the top surface of the fingerprint sensor.

3. The fingerprint identification module according to claim 1, wherein before the covering plate is combined with the fingerprint sensor, the solid adhesive part is attached on a top surface of the fingerprint sensor, there is a gap between the solid adhesive part and a border of the top surface of the fingerprint sensor, the liquid adhesive part is formed on a bottom surface of the covering plate, and the liquid adhesive part is formed on a periphery region of the bottom surface of the covering plate.

4. The fingerprint identification module according to claim 1, wherein before the covering plate is combined with the fingerprint sensor, the solid adhesive part and the liquid adhesive part are both attached on a top surface of the fingerprint sensor, there is a gap between the solid adhesive part and a border of the top surface of the fingerprint sensor, and the liquid adhesive part is disposed within the gap.

5. The fingerprint identification module according to claim 1, wherein before the covering plate is combined with the fingerprint sensor, the solid adhesive part and the liquid adhesive part are both attached on a bottom surface of the covering plate, there is a gap between the solid adhesive part and a border of the bottom surface of the covering plate, and the liquid adhesive part is disposed within the gap.

6. The fingerprint identification module according to claim 1, wherein a distance between the solid adhesive part and a border of a bottom surface of the covering plate or a distance between the solid adhesive part and a border of a top surface of the fingerprint sensor is in a range between 1 mm and 2 mm.

7. The fingerprint identification module according to claim 1, wherein the substrate is a flexible circuit board, or the fingerprint sensor is a fingerprint sensing chip, or the covering plate is a glass plate or a ceramic plate, or the solid adhesive part is a film adhesive, or the liquid adhesive part is a water-based adhesive.

8. A manufacturing method of a fingerprint identification module, the manufacturing method comprising steps of:
(a) forming a solid adhesive part on a first one of a covering plate and a fingerprint sensor, and forming a liquid adhesive part on a second one of the covering plate and the fingerprint sensor, wherein there is a gap between the solid adhesive part and the first one of the covering plate and the fingerprint sensor, and the liquid adhesive part is formed on a periphery region of the second one of the covering plate and the fingerprint sensor; and
(b) moving the covering plate toward the fingerprint sensor, or moving the fingerprint sensor toward the covering plate, so that the covering plate and the fingerprint sensor are combined together and the liquid adhesive part is arranged around the solid adhesive part.

9. The manufacturing method according to claim 8, wherein the gap is in a range between 1 mm and 2 mm.

10. The manufacturing method according to claim 8, wherein the substrate is a flexible circuit board, or the fingerprint sensor is a fingerprint sensing chip, or the covering plate is a glass plate or a ceramic plate, or the solid adhesive part is a film adhesive, or the liquid adhesive part is a water-based adhesive.

11. A manufacturing method of a fingerprint identification module, the manufacturing method comprising steps of:
(a) forming a solid adhesive part and a liquid adhesive part on a specified one of a covering plate and a fingerprint sensor, wherein there is a gap between the solid adhesive part a border of the specified one of the covering plate and the fingerprint sensor, and the liquid adhesive part is disposed within the gap and arranged around the solid adhesive part; and
(b) moving the covering plate toward the fingerprint sensor, or moving the fingerprint sensor toward the covering plate, so that the covering plate and the fingerprint sensor are combined together.

12. The manufacturing method according to claim 11, wherein the gap is in a range between 1 mm and 2 mm.

13. The manufacturing method according to claim 11, wherein the substrate is a flexible circuit board, or the fingerprint sensor is a fingerprint sensing chip, or the covering plate is a glass plate or a ceramic plate, or the solid adhesive part is a film adhesive, or the liquid adhesive part is a water-based adhesive.
